(19) United States
(12) Patent Application Publication
(10) Pub. No.: US 2013/0190582 A1
(43) Pub. Date: Jul. 25, 2013

(54) ISCHEMIC REGION DETECTION

(71) Applicants: Nils Holmstrom, Jarfalla (SE); Michael Broome, Ekero (SE)

(72) Inventors: Nils Holmstrom, Jarfalla (SE); Michael Broome, Ekero (SE)

(21) Appl. No.: 13/358,063

(22) Filed: Jan. 25, 2012

(51) Int. Cl. A61B 5/145; A61B 5/02

(52) U.S. Cl. 600/364; 600/309

(57) ABSTRACT

An ischemia detecting system has an implantable medical device connectable to an LV cardiac catheter with sensors for generating sensor signals representative of the concentration of a constituent in coronary venous blood at different sites in the coronary venous system. The sensor signals are co-processed by the system to detect an ischemic region of the subject's heart based on a relation between the sensor signals. The detection of the particular ischemic cardiac region is possible by conducting the concentration monitoring on either sides of a branching vein in the coronary venous system and co-processing the sensor signals.

### BACKGROUND OF THE INVENTION

1. Field of the Invention

The present invention generally relates to ischemia detection, and in particular to detection of ischemic heart regions based on constituent concentration measurements in coronary venous blood.

2. Description of the Prior Art

Ischemia is the lack of oxygen supply to cells. In animals, including humans, the underlying cause of ischemia is typically a cardiovascular disease, where blood vessels may be affected by atherosclerosis. Cardiac ischemia is caused by restriction of blood flow in the coronary blood vessel, e.g. due to atherosclerosis. This reduced blood flow and the resulting lack of oxygen to the myocytes in the heart may lead to several effects, including contractile dysfunction and hibernating cells. These various effects may in turn decrease the hemodynamic performance of the heart, which ultimately can result in worsening heart failure and further decrease in pumping capacity.

Ischemic heart disease (IHD) is very common. IHD may be symptomatic, such as in angina pectoris, causing the patient to experience severe discomfort and pain. However, a majority of ischemic periods are silent and therefore hard to detect and classify. Most ischemic episodes, regardless of being symptomatic or silent, are reversible but still influence the risk of arrhythmias, the functional state and long-term remodeling of the heart.

A common technique in the art to detect cardiac ischemia is to measure oxygen saturation (SO₂) in coronary sinus blood, such as disclosed in U.S. Pat. No. 5,156,148; U.S. Pat. No. 5,199,428; US 2005/0154370; US 2008/0177194 and EP 1 386 637. These prior art documents generally state that cardiac ischemia is detected as a decrease in SO₂ in the coronary sinus blood.

The prior art techniques basically operate according to an on-off principle. In other words, they either detect presence of an ischemic event or they conclude that there is no detectable ischemic event. However, it is generally preferred to be able to obtain a more accurate detection of the particular heart region where the ischemic event takes place. Such detailed ischemic analysis is not possible or at least very hard with to the above mentioned prior techniques.

### SUMMARY OF THE INVENTION

It is a general objective to enable detection of the particular region of a heart that suffers from an ischemic event.

This and other objectives are met by embodiments as disclosed herein.

An aspect of the embodiments relates to an ischemia detecting system comprising an implantable medical device comprising a connector. The connector is connectable to a left ventricular cardiac catheter equipped with at least two sensor elements arranged on the catheter to be positioned on either side of a branching vein in the coronary venous system of a subject's heart. A first sensor signal is generated for the first sensor element to be representative of the concentration of a constituent in coronary venous blood in a first portion of the coronary venous system. A corresponding second sensor signal is generated for the second sensor element to be representative of the concentration of the constituent in coronary venous blood in a second, different portion of the coronary venous system. The sensor signals are stored in a memory of the implantable medical device and are co-processed by an ischemic region detector. The ischemic region detector is configured to detect an ischemic heart region based on a relation between the sensor signals.

Another aspect of the embodiments defines a method of detecting ischemia. The method comprises recording a first sensor signal representative of the concentration of a constituent in coronary venous blood in a first portion of the coronary venous system of a subject's heart at a site on a first side of a branching vein of the coronary venous system. A second sensor signal representative of the concentration of the constituent in coronary venous blood is recorded for a second portion of the coronary venous system at a site on a second, opposite side of the branching vein. The sensor signals are then co-processed to detect an ischemic region of the heart based on a relation between the sensor signals.

The embodiments are thereby able to not only detect the presence of cardiac ischemia but are also able to identify the particular region of the heart where the ischemia has occurred. This provides valuable diagnostic information and can also be used as a basis for more effective treatment.

### BRIEF DESCRIPTION OF THE DRAWINGS

FIG. 1 is a schematic overview of a subject and an ischemia detecting system according to an embodiment.

FIG. 2 is a schematic block diagram of an ischemia detecting system according to an embodiment.

FIG. 3 is a schematic block diagram of an ischemia detecting system according to another embodiment.

FIG. 4 is a drawing of the coronary venous system of a human heart.

FIG. 5 is a flow diagram illustrating a method for detecting ischemia according to an embodiment.

FIG. 6 is a diagram illustrating concentrations of a blood-borne constituent at various positions in a coronary venous system prior to and after an ischemic event.

### DESCRIPTION OF THE PREFERRED EMBODIMENTS

The embodiments generally relate to ischemia detection, and in particular to detection of ischemic heart regions based on constituent concentration measurements in coronary venous blood or, as it is sometimes denoted, coronary sinus blood.

The ischemia detection of the embodiments enables identification of the particular heart region where an ischemic event has occurred. Hence, the embodiments enable pinpointing the particular region in the heart that has suffered from ischemia. The detailed information of the particular ischemic heart region constitutes valuable diagnostic information to the patient's physician in determining appropriate therapy to the patient and detecting any trend or worsening heart condition that may subsequently lead to severe conditions, such as myocardial infarction or heart failure. The localization of the ischemic heart region is also of value in selecting a particular pacing mode for a pacemaker or implantable cardioverter-defibrillator (ICD) of the patient.

The embodiments therefore provide a significant improvement to the prior art ischemia detecting systems that basically only are able to detect the presence or absence of cardiac ischemia but without any further detailed information of in which part of the heart the ischemia has occurred.

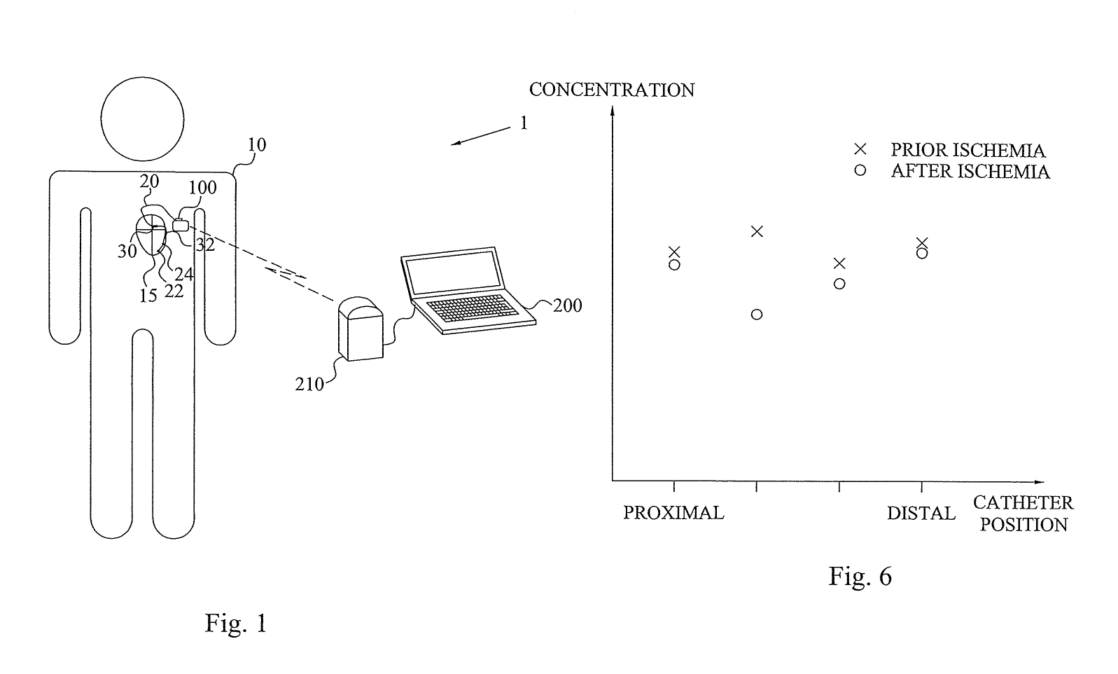

FIG. 1 is a schematic overview of an embodiment of an ischemia detecting system 1 and a subject, represented by a human subject 10 having an implantable medical device (IMD) 100 according to the embodiments. The IMD 100 is implanted in the subject 10 in order to detect ischemic episodes in the subject's heart 15. The IMD 100 can, in addition, be employed to provide cardiac therapy and/or monitoring, such as to provide pacing therapy to the subject's heart 15. In such a case, the IMD 100 can be in form of a pacemaker or an ICD. The IMD 100 is, however, not limited to such device implementations but can indeed be an implantable ischemia detecting device or be any implantable medical device that is equipped with an ischemia detecting functionality as disclosed herein.

The IMD 100 is in operation connected to a left ventricular (LV) cardiac catheter or lead 20 comprising multiple, i.e. at least two, sensor elements 30, 32 arranged at different positions on the LV cardiac catheter 20. These sensor elements 30, 32 are employed by the ischemia detecting system 1 in order to monitor the concentration of a selected constituent in coronary venous blood in different parts of the coronary venous system of the heart 15.

The LV cardiac catheter 20 could be a dedicated sensor-carrying catheter. Alternatively, the LV cardiac catheter 20 not only carries the sensor elements 30, 32 but is also used by the IMD 100 for providing cardiac therapy and/or monitoring. For instance, the LV cardiac catheter could be an LV cardiac lead 20 having at least one electrode 22, 24, typically denoted pacing and sensing electrode 22, 24 in the art. In such a case, the IMD 100 is able to deliver pacing pulses to the left ventricle of the heart 15 and sense electrical activity from the left ventricle via the electrode(s) 22, 24 of the LV cardiac lead 20. As is well known in the art, an LV cardiac lead 20 is generally implanted via the coronary venous system. Hence, the LV cardiac lead 20 is therefore highly suitable to carry the sensor elements 30, 32 to position these sensor elements 30, 32 at different positions within the coronary venous system to enable monitoring of the constituent concentration in coronary venous blood.

The IMD 100 can also be connected to other cardiac leads, for instance a right ventricular (RV) cardiac lead and/or an atrial cardiac lead (not illustrated). An RV cardiac lead is typically provided inside the right ventricle of the heart 15 and comprises one or more electrodes that can be used by the IMD 100 to apply pacing pulses to the right ventricle and/or sense electrical activity from the right ventricle. An atrial cardiac lead, typically a right atrial (RA) cardiac lead having at least one electrode arranged in or in connection with the right atrium, can be used by the IMD 100 in order to provide atrial pacing and/or sensing. Instead of or as a complement to an RA cardiac lead, the IMD 100 can be connected to a left atrial (LA) cardiac lead.

FIG. 1 additionally illustrates a non-implantable data processing device 200, such as in the form of a programmer, a home monitoring device, a hand-held or portable device, or a physician's workstation. The data processing device 200 comprises or is connected to a communication module or device 210 that is capable of wirelessly communicating with the IMD 100, preferably through radio frequency (RF) based communication or inductive telemetry. The data processing device 200 uses the communication module 210 in order to interrogate the IMD 100 for diagnostic data recorded by the IMD 100 employing the electrodes 22, 24 of the connected cardiac lead(s) 20 and/or at least one sensor element 30, 32 generating sensor signals representing the concentrations of the selected constituent in coronary venous blood. Furthermore, the data processing device 200 can be used to program the IMD 100, such as by setting one or more programmable pacing parameters. According to the present embodiments, the IMD 100 can in particular transmit notifications of sensor signals to the data processing device 200 for processing therein to detect the ischemic region of the heart 15 and preferably display to the subject 10 or the subject's physician information of this detected heart region.

The communication module 210 and the data processing device 200 can be separate devices as illustrated in FIG. 1, either wired connected or using a wireless connection, such as Bluetooth®, an infrared (IR) connection or an RF connection. In an alternative embodiment, the functionality and equipment of the communication module 210 can be housed within the data processing device 200.

In a general aspect of the embodiments an ischemia detecting system comprises an implantable medical device comprising a connector that is connectable to an LV cardiac catheter comprising at least a first sensor element and a second sensor element arranged on the LV cardiac catheter. The sensor elements are provided on the LV cardiac catheter to be positioned on either side of a branching vein in the coronary venous system of the subject's heart when the LV cardiac catheter is implanted in the coronary venous system. The first sensor element and the second sensor element either constitute a respective sensor, i.e. the LV cardiac catheter comprises at least a first sensor and a second sensor, or the at least two sensor elements are connectable to a common sensor of the ischemia detecting system. In the former case, the first sensor is configured to generate a first sensor signal representing a concentration of a selected constituent in coronary venous blood in a first portion of the coronary venous system. The second sensor is correspondingly configured to generate a second sensor signal representing a concentration of the constituent in coronary venous blood in a second, different portion of the coronary venous system. In the latter case the common sensor is configured to generate the first sensor signal for the first sensor element and generate the second sensor signal for the second sensor element. The implantable medical device also comprises a memory configured to store the first and second sensor signals. The ischemia detecting system comprises an ischemic region detector configured to co-process the first sensor signal and the second sensor signal to detect an ischemic region of the heart based on a relation between the first sensor signal and the second sensor signal.

Thus, by conducting the constituent concentration measurements at different sites in the coronary venous system and having the sensor elements present on either sides of a branching vein in the coronary venous system, the ischemia detecting system can detect a particular region of the heart that is suffering from ischemia by co-processing the sensor signals and in particular based on a relation between the sensor signals.

For instance, if the first (or second) sensor signal recorded by the first (or second) sensor or the common sensor for the first (or second) sensor element indicates a significant change in the concentration of the constituent while the other sensor signal does not indicate any change or at least a much lesser change in the concentration of the constituent it is possible to locate an ischemic region to be present at a portion of the heart between the positions of the two sensor elements.

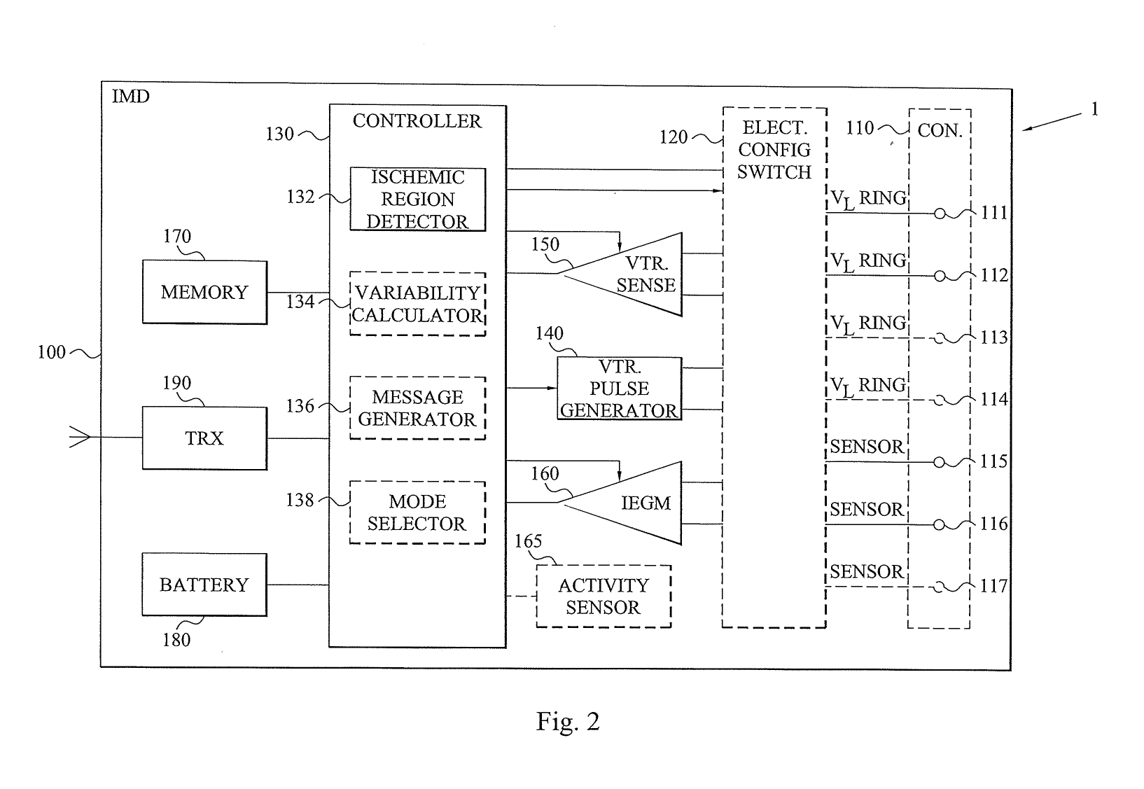

FIG. 2 is a schematic block diagram of an ischemia detecting system 1 according to an embodiment. In this embodiment, the ischemia detecting system 1 is a fully implantable system housed within an IMD 100.

The IMD 100 comprises a connector 110 connectable to the LV cardiac catheter and therefore comprises connector terminals 115-117 configured to be connected to the sensor elements of the LV cardiac catheter. In FIG. 2, the connector 110 has been exemplified as comprising three such connector terminals 115-117 to be used with an LV cardiac catheter having three sensor elements. This should, however, merely be seen as an illustrative but non-limiting example. The embodiments are to be used with an LV cardiac catheter comprising at least two sensor elements.

In an embodiment, each sensor element constitutes a respective sensor. Hence, the LV cardiac catheter then comprises at least a first sensor and a second sensor arranged on the LV cardiac catheter to be positioned on either side of the branching vein in the coronary venous system. The first sensor then generates the first sensor signal representing the concentration of the selected constituent in coronary venous blood in the first portion of the coronary venous system and the second sensor generates the second sensor signal representing the concentration of the selected constituent in coronary venous blood in the second portion of the coronary venous system. The connector 110 then preferably comprises a respective connector terminal 115-117 for each of the sensors along the LV cardiac catheter.

In another embodiment, the LV cardiac catheter comprises a common sensor that is connected to each sensor element. The common sensor could then be positioned anywhere along the LV cardiac catheter but is typically proximally arranged relative to the sensor elements, i.e. closer to the end of the LV cardiac catheter that is to be connected to the IMD 100 and the connector 110 as compared to the positions of the sensor elements on the LV cardiac catheter. Each sensor element then comprises at least one respective sensor connection to the common sensor. The common sensor generates the sensor signals for each of the sensor elements and forwards them to the connector 110. In such a case, it could be sufficient to only include a single connector terminal 115-117 in the connector 110 that is connectable to the common sensor, although multiple parallel connector terminals 115-117 to the common sensor are indeed possible.

A further embodiment houses a common sensor not on the LV cardiac catheter but rather within the housing of the IMD 100. Each sensor element on the LV cardiac catheter then preferably comprises at least one respective connection to the common sensor through at least one respective connector terminal 115-117 in the connector 110. The common sensor (not illustrated) in the IMD 100 then generates the sensor signals for the different sensor elements.

The selected constituent which is monitored by the at least one sensor can be any substance or molecule present in coronary venous blood and that can be used for ischemia detection. A particular preferred example of such constituent is to monitor oxygen concentration in coronary venous blood. The at least one sensor is then an implantable oxygen sensor. The implantable oxygen sensor does not necessarily have to be able to produce a sensor signal that represents an absolute oxygen concentration value. It is sufficient if the implantable oxygen sensor can be used to monitor variations in oxygen concentration and thereby produce a sensor signal that represents relative oxygen concentration values.

There are various implantable oxygen sensors disclosed in the art and that can be used according to the embodiments. For instance, the implantable oxygen sensor can be a partial oxygen pressure (pO₂) sensor, such as an electrochemical pO₂ sensor, or a sensor that measures oxygen saturation (SO₂), such as an optical SO₂ sensor, or indeed any other type of implantable sensor that outputs a sensor signal that represents variations of oxygen concentration in venous blood in the coronary venous system.

In an embodiment, the implantable oxygen sensor could use a measurement sampling frequency to generate an oxygen concentration sample every heartbeat, or multiple such samples per heartbeat. Also a low sampling frequency could be used, such as every second heart beat or even lower. Instead of basing the sampling frequency on the heart rate, the implantable oxygen sensor could generate a measurement sample once every 0.1-10 seconds as illustrative but non-limiting examples.

In an embodiment, the LV cardiac catheter comprises multiple oxygen sensors each generating a respective sensor signal as previously discussed herein. The oxygen sensors could then, for instance, be electrochemical pO₂ sensors or indeed optical SO₂ sensors. If a common oxygen sensor is to be used, either arranged on the LV cardiac catheter or inside the housing of the IMD 100, the common oxygen sensor is preferably an optical SO₂ sensor. In such a case, one or a pair of optical fibers runs from the common optical SO₂ sensor up to each sensor element along the LV cardiac catheter. Light with a particular wavelength is produced by the common SO₂ sensor, such as a diode of the common SO₂ sensor, and guided to each sensor element by a respective optical fiber. Returning light is then forwarded by these optical fibers or the other optical fibers in the case of a pair of optical fibers per sensor element back to the common SO₂ sensor for generating the sensor signals.

Alternatively other substances besides oxygen could be monitored and used by the ischemia detecting system 1. Examples of such other substances include nitric oxide (NO) and carbon dioxide (CO₂) in coronary venous blood.

The connector 110 may additionally comprise connector terminals 111-114 configured to be connected to matching electrode terminals electrically connected to electrodes on one or more cardiac leads. With reference to FIGS. 1 and 2, in the particular shown example, the connector 110 comprises two connector terminals 111, 112, each of which is electrically connectable to a respective electrode terminal of the LV cardiac lead 20 and the electrodes 22, 24 of the LV cardiac lead 20. In this example, the LV cardiac lead 20 is a so-called bipolar cardiac lead, i.e. having two electrodes 22, 24. However, this should merely be seen as an illustrative example and the LV cardiac lead 20 can in fact comprise zero, one, two, three, four or more electrodes 22, 24. The connector 110 then comprises a respective connector terminal 111, 112 for each of the electrodes 22, 24. FIG. 2 schematically indicates that the LV cardiac lead 20 could be a so-called quadropolar lead with four electrodes 22, 24. In such a case, the connector 110 comprises four connector terminals 111, 112, 113, 114 to electrically connect the IMD 100 to these four electrodes 22, 24.

The IMD 100 also comprises a memory 170 that is configured to store the first and second sensor signals. The memory 170 is therefore directly, or as indicated in FIG. 2 indirectly through an optional electrical configuration switch 120, connected to the connector 110 for receiving the sensor signals therefrom. If the IMD 100 comprises the common sensor as discussed in the foregoing the memory 170 is preferably connected to this common sensor.

In the embodiment illustrated in FIG. 2, the IMD 100 comprises an ischemic region detector 132 configured to co-process the first and second sensor signal, typically as fetched from the memory 170. In particular, the ischemic region detector 132 is configured to detect an ischemic heart region based on a relation between the first sensor signal and the second sensor signal.

In an embodiment, the ischemic region detector 132 performs the co-processing based on a relation between the concentration of the selected constituent in coronary venous blood in a first portion of the heart as represented by the first sensor signal and the concentration of the constituent in coronary venous blood in a second portion of the heart as represented by the second sensor signal.

The co-processing of the first and second sensor signal can be based on calculating a respective average concentration value with regard to the selected constituent in coronary venous blood. Thus, each sensor signal is preferably in the form of a series of signal samples having a respective sample value representing the concentration of the constituent in coronary venous blood. In such a case, the average concentration value can be calculated based on a defined number of, preferably consecutive, signal samples in the sensor signal or based on multiple, preferably consecutive, signal samples in the sensor signal recorded during a defined period of time. The relation between these average concentration values obtained from the first and second sensor signals are then preferably used by the ischemic region detector 132 to identify the correct region of the heart where ischemia has occurred. Averaging the concentration suppresses noise and other disturbing effects in the sensor signals that might occur due to temporary phenomena that are not related to ischemia.

An example of co-processing is to compare a quotient or difference between the (average) constituent concentrations obtained from the sensor signals with a threshold value. This threshold value is then preferably stored in the memory 170 and represents a previously calculated quotient or difference between the (average) constituent concentrations obtained from the sensor signals at a point in time when the heart was not suffering from ischemia. Thus, assume that C₁ represents the (average) concentration of the constituent in coronary venous blood in the first portion of the heart represented by the first sensor signal and C₂ represents the (average) concentration of the constituent in coronary venous blood in the second portion of the heart represented by the second sensor signal. The ischemic region detector 132 could compare

$$\frac{{C}_{1}}{{C}_{2}} ( or \frac{{C}_{2}}{{C}_{1}} )$$

with the threshold value T₁ or compare (C₁−C₂) (or (C₂−C₁)) with the threshold value T₂.

For instance, in a non-ischemic condition the quotient

$$\frac{{C}_{1}}{{C}_{2}}$$

is typically equal to or at least close to a normal or default value, represented by the threshold value T₁. However, if an ischemic event occurs in the second heart region (but does not affect the first heart region) the coronary venous blood flow past the second sensor element will drop significantly, thereby reducing the amount of the selected constituent that is detected via the second sensor element. The average concentration value C₂ will thereby drop significantly. As a result the quotient

$$\frac{{C}_{1}}{{C}_{2}}$$

will increase significantly above the threshold value T₁, i.e. be larger than T₁+ΔT where ΔT represents a hysteresis margin to indicate that the normal value of the quotient can fluctuate within the interval T₁±ΔT during normal, non-ischemic conditions.

A significant rise in the quotient thereby indicates that an ischemic event has occurred in a region of the heart that affects the concentration of the constituent in the coronary venous blood at the position of the second sensor element but does not significantly affect the concentration of the constituent at the position of the first sensor element in the coronary venous system. Correspondingly, a significant drop in the quotient indicates that an ischemic event has occurred in a region of the heart that affects the concentration of the constituent in the coronary venous blood at the position of the first sensor element but does not significantly affect the concentration of the constituent at the position of the second sensor element in the coronary venous system.

A somewhat smaller rise or drop in the quotient also provides valuable information by indicating that the ischemic event in particular affects the second heart region (in the case of a small rise) or the first heart region (in the case of a small drop) but also, but to a lesser extent, affects the concentration of the constituent in the first heart region (in the case of a small rise) or the second heart region (in the case of a small drop).

FIG. 6 visually illustrates another embodiment of co-processing the sensor signals that is particularly suitable if more than two sensor elements are available and more than two sensor signals are recorded for the subject's heart. In the example shown in FIG. 6 the LV cardiac catheter comprises four sensor elements arranged at different positions along the length of the catheter. These four positions are indicated in FIG. 6 starting from a most proximal sensing position with two intermediate sensing positions and a most distal sensing position. At normal healthy conditions the (average) concentrations of the selected constituent at the different sensing positions in the coronary venous system are as indicated by the crosses in FIG. 6. The circles indicate the corresponding (average) concentrations of the constituent at the sensing positions during ischemia. As is seen in FIG. 6, the concentration drops significantly at the second most proximal sensing position and to a lesser extent at the second most distal sensing position and at the most distal sensing position. There is basically no difference in measured concentration at the most proximal sensing position. This co-processing of the sensor signals enable determining that the ischemia does not significantly affect the blood flow at the most proximal sensing position but clearly restricts the blood flow in the part of the coronary venous system where the second most proximal sensing element is present. The restricted blood flow also partly effects the more distal sensing elements but this effect decreases the further away one travels in the coronary venous system from the second most proximal sensing position. This means that sufficient blood flow is still present at the two most distal sensing positions.

This information can be used to accurately pinpoint and detect the region of the heart where the ischemia is present. This is possible since the positions of the different sensing elements along the LV cardiac catheter is known and also the particular vein of the coronary venous system in which the LV cardiac catheter is present.

In an embodiment, the IMD 100 comprises a variability calculator 134, see FIG. 2, configured to calculate a first variability value based on the first sensor signal. This first variability value represents a variability in the concentration of the constituent in coronary venous blood in the first portion of the heart represented by the first sensor signal. The variability calculator 134 also calculates a second (and further) variability value based on the second (or further) sensor signal. The second (and further) variability value represents the variability in the concentration of the constituent in coronary venous blood in the second (or further) portion of the heart represented by the second (or further) sensor signal. The ischemic region detector 132 may then be configured to detect the ischemic heart region based on a relation between the first variability value and the second (and further) variability value.

During an occlusion in a blood vessel of the coronary venous system causing a local ischemia the variability in the concentration of the constituent will significantly increase as compared to during normal, healthy conditions. Hence, co-processing in the form of performing the detection based on the relation between variability values can be used according to the embodiments.

In connection with a temporary ischemic event, there is generally a variation in oxygen concentration in coronary venous blood. First, if there is an occlusion of a blood vessel, thereby reducing the blood flow there is an initial decrease in oxygen concentration in coronary venous blood. Correspondingly, if there is a mismatch between the demands for and the supply of oxygen to a portion of the heart ischemia may occur and is initially detectable as reduction in oxygen concentration in coronary venous blood. Such a mismatch between oxygen supply and demand can be due to occlusions in blood vessels, increased oxygen consumption by the myocytes and/or local coagulation disturbances. Secondly, following the temporary ischemic event there is a reperfusion causing an increase in oxygen concentration above the normal levels in coronary venous blood. Hence, a temporary ischemic event generally causes an initial reduction in oxygen concentration below the normal or baseline level followed by a temporary increase in oxygen concentration above the normal level and then a reduction of the oxygen concentration back to the normal level.

If the ischemia detecting system 1 is configured to generate sensor signals representing the concentration of oxygen in coronary venous blood from various regions of the coronary venous system the co-processing of the sensor signals does not necessarily have to be in the form of detecting a significant decrease in oxygen concentration at one or more of the monitoring sensor element sites. It could actually be advantageous to detect a local ischemic region by an initial and temporary decrease in oxygen concentration for that site below the normal baseline concentration followed by an increase in oxygen concentration above the normal baseline concentration and then return back to the normal baseline concentration. Such an approach improves sensitivity in the ischemia detection and the determination of the ischemic heart region as compared to base the detection and determination solely on a decrease in oxygen concentration, i.e. without any following, temporary increase in oxygen concentration above the baseline concentration. The reason is that certain events, such as increased patient activity, can cause a temporary decrease in oxygen concentration although no cardiac ischemia is present. However, such activity-induced temporary reductions in oxygen concentrations generally return back to the baseline concentration without any intermediate overshoot or increase in oxygen concentration before the return to the baseline concentration.

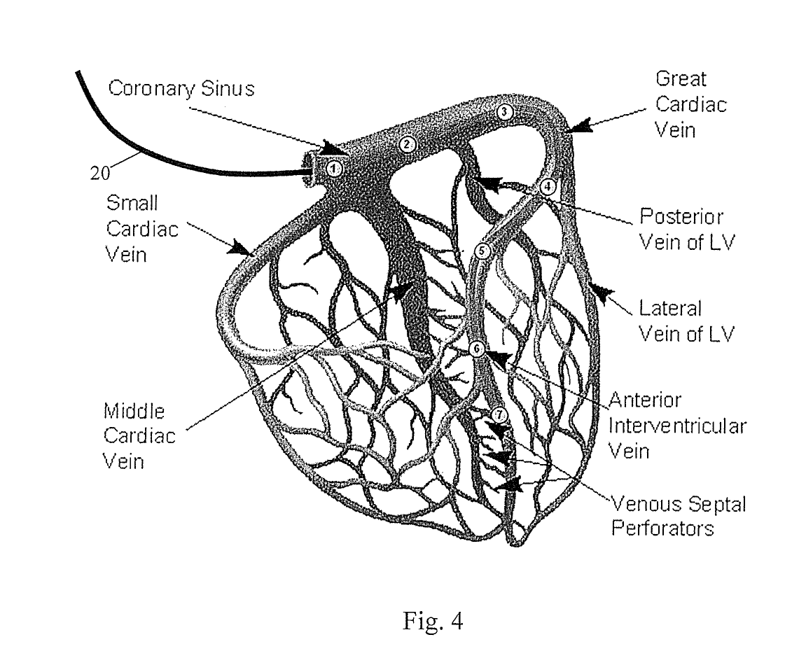

FIG. 4 is a schematic overview of the coronary venous system of a human heart. In an embodiment, the LV cardiac catheter 20 is configured to be implanted into the posterior vein of the left ventricle. In such a case, the first sensor element of the LV cardiac catheter 20 is preferably arranged on the catheter to be positioned in the coronary sinus whereas the second sensor element is preferably arranged on the catheter to be positioned in the posterior vein. The second sensor element monitors the posterior wall of the heart, while the first sensor element monitors the region of the heart in connection with the valve plane and the anterior wall.

In another embodiment, the LV cardiac catheter 20 is implanted into the lateral vein of the left ventricle. At this position in the coronary venous system the first sensor element is preferably positioned in the coronary sinus or in the great cardiac vein while the second sensor element is positioned in the lateral vein. With such an implantation site for the LV cardiac catheter 20 it could be beneficial to have more than two sensor elements along the LV cardiac catheter 20. Thus, a third sensor element is preferably arranged on the LV cardiac catheter 20 in addition to the first and second sensor elements. This third sensor element can constitute a third sensor or is connectable to the common sensor arranged proximally on the LV cardiac catheter 20 or inside the housing of the IMD. The third sensor or the sensor connected to the third sensor element then generates a third sensor signal representing a concentration of the constituent in coronary venous blood in a third portion of the coronary venous system. The first sensor element is preferably arranged on the LV cardiac catheter 20 to be positioned in the coronary sinus upstream of the posterior vein of the left ventricle and with the third sensor element configured to be positioned in the coronary sinus or the great cardiac vein but downstream of the posterior vein.

The memory 170 of the IMD 100, see FIG. 2, then stores the first, second and third sensor signals and the ischemic region detector 132 co-processes these sensor signals to detect the ischemic region of the heart based on a relation between the sensor signals or between (average) concentration values calculated based on the sensor signals.

In another embodiment, the LV cardiac catheter 20 is configured to be implanted in the anterior interventricular vein of the heart. The first sensor element could be arranged on the catheter to be position in the coronary sinus or in the great cardiac vein whereas the second sensor element could be arranged on the catheter to be positioned in the anterior interventricular vein. Also this embodiment could benefit from having at least one additional sensor element in addition to the first and second sensor element. The first sensor element is then preferably positioned in the coronary sinus upstream of the posterior vein of the left ventricle and the third sensor element is positioned in the coronary sinus or in the great cardiac vein downstream of the posterior vein. Alternatively, the first sensor element is positioned in the coronary sinus or in the great cardiac vein upstream of the lateral vein of the left ventricle and the third sensor element is positioned in the great vein downstream of the lateral vein. These embodiments may in fact be combined by having access to more than three sensor elements along the LV cardiac vein. Sensor elements can then be present in the coronary sinus or in the great cardiac vein at positions selected among upstream of the small and middle cardiac vein, downstream of the small and middle cardiac vein but upstream of the posterior vein of the left ventricle, downstream of the posterior vein of the left ventricle but upstream of the lateral vein of the left ventricle, and downstream of the lateral vein of the left ventricle. In addition, or alternatively different sensing positions are possible along the anterior interventricular vein. FIG. 4 schematically indicates seven suitable positions that can be used for sensor elements on the LV cardiac catheter.

As is evident from the discussion presented above, in a general embodiment the connector 110 of the ischemia detecting system 1 in FIG. 2 is connectable to the LV cardiac catheter comprising N sensor elements arranged on the LV cardiac catheter to be positioned at different sites in the coronary venous system. The N sensor elements constitute a respective sensor or is each connected to a common sensor in the LV cardiac catheter or connectable to a common sensor in the IMD 100. A sensor signal representative of the concentration of the constituent in coronary venous blood in a respective portion of the coronary venous system is generated for each sensor element of the N sensor elements. In a general embodiment the parameter N is an integer equal to or larger than two and in a particular embodiment, the parameter N is equal to or larger than three.

The memory 170 is configured to store these N sensor signals and the ischemic region detector 132 is configured to co-process the N sensor signals as disclosed herein to detect the ischemic region of the heart based on a relation between the N sensor signals.

In a particular embodiment the ischemia detecting system 1 comprises an activity sensor 165 preferably provided inside the housing of the IMD 100 but can alternatively be attached to the outside surface of the housing, be arranged on the LV cardiac catheter or otherwise connected to the IMD 100 through the connector 110.

The activity sensor 165 is configured to generate an activity signal representative of a current activity level of the subject. The ischemic region detector 132 is then responsive to the activity signal and is configured to perform the co-processing of the sensor signals to detect the ischemic heart region if the current activity level of the subject as defined by the activity signal exceeds an activity threshold level. Thus, in this embodiment the ischemia region detecting operation of the ischemia detecting system 1 is only performed when the subject is active and exercises at a level that corresponds at least to the activity threshold level, which is advantageously defined in the memory 170.

A reason for limiting the ischemia region detection to active periods for the subject is that certain ischemic events, in particular rather mild ischemic episodes, only appears and are detectable if the subject is active, such as up and walking.

As an alternative to limiting the ischemia region detection to active periods, the ischemic region detector 132 could perform the co-processing of the sensor signals and the ischemic heart region detection regardless of the current activity level of the subject. However, information of the detected ischemic heart region, such as represented by information of which sensor element(s) that has(have) indicated a significant change in the constituent concentration in coronary venous blood, could be tagged with information of the current activity level, i.e. is associated with a current, optional average, activity signal value.

The activity level information can then be used together with information of the ischemic heart region by the physician to determine any trends in the condition of the heart and select appropriate combating actions if a deterioration is evident.

The ischemic region detector 132 generates, in an embodiment, a notification of the detected heart region. This notification could be a simple identifier of the sensor elements (or sensors) detecting a significant change in the concentration of the constituent in coronary venous blood. Alternatively the memory 170 is preprogrammed with information of, for each sensor element, which heart region(s) that get its(their) blood supply from particular blood vessel in which the sensor element is present. In such a case, the notification can comprise information of such a detected heart region. The notification may additionally comprise information of the current activity level of the subject as discussed in the foregoing.

The IMD 100 could then comprise a message generator 136 configured to generate a notification message comprising the notification of the detected ischemic heart region. The message generator 136 could be configured to generate the notification message in response to the ischemic region detector 132 detecting the ischemic heart region, i.e. basically generating the notification directly following the ischemia detection. The notification message could then be regarded as an alarm message that will inform the subject or the subject's physician in real time of any ischemic periods and regions. The notification message is wirelessly transmitted by a transmitter or transceiver (TRX) 190 to the data processing device (see FIG. 1). If the data processing device is in the form of a home monitoring device or a hand-held device carried by the subject, the device will inform the subject of the immediate ischemic event and region. This information can be used by the subject to, for instance, contact his/her physician and/or guide the subject in optimizing drug administration or planning of revascularization procedures, such as coronary vascular surgery or percutaneous stent implantations. The information could also be of value for the subject to optimize his/her lifestyle in general to avoid episodes of ischemia.

The IMD 100 could alternatively store the notifications and any additional information descriptive of the detected ischemic events in the memory 170 to form a register of the subject's ischemia burden. The collected information is then uploaded when the patient visits his/her physician and is used as diagnostic information and/or for determination of optimal drug titration, device settings for the subject or planning revascularization procedures.

In addition to or as a complement to generating notifications of detected ischemic events, the IMD 100 could initiate cardiac therapy to combat or compensate for the ischemic event. The IMD 100 then comprises a pulse generator 140, represented by a ventricular pulse generator 140 in FIG. 2. The pulse generator 140 is connected to the connector 110, optionally through the electronic configuration switch 120. The pulse generator 140 is configured to generate pacing pulses to be applied, through the optional switch 120 and the connector 110, to at least one of the connected cardiac leads and one or more electrodes of the cardiac leads.

The pulse generator 140 is connected to and controlled by a pulse generator controller 130, represented by a general controller 130 in FIG. 2. The pulse generator controller 130 controls the particular pacing pattern to be applied to the subject's heart. In a particular embodiment the pulse generator controller 130 is configured to control the pulse generator 140 to generate and apply pacing pulses to connected electrodes according to either a default pacing mode or an ischemic pacing mode. The pulse generator controller 130 is thereby configured to switch from the default or normal pacing mode to the ischemic pacing mode based on the ischemia detector 132 detecting an ischemic event and heart region.

The default pacing mode is the traditional pacing mode selected for the subject by the physician based on the particular cardiac disease of the subject. This means that the pulse generator controller 130 mainly operates according to this default pacing mode. However, in connection with a detected ischemic heart region as signaled by an ischemia signal generated by the ischemic region detector 132 in response to the detection of an ischemic heart region, the pulse generator controller 130 switches to the ischemic pacing mode. The controller 130 could include a dedicated mode selector 138 that is responsive to the ischemia signal and triggers a switch to the ischemia pacing mode based on the ischemia signal.

Compared to the default pacing mode, the ischemic pacing mode could provide a reduced load on the myocardium of the heart, for instance by reducing the maximum tracking rate or the rate response function used in the default pacing mode. Furthermore, or alternatively, the IMD 100 could be configured to be more sensitive to any arrhythmia events when operating in the ischemic pacing mode as compared to the default pacing mode. Generally, the IMD 100 comprises an intracardiac electrogram (IEGM) processor or circuit 160 connected to the connector 110 and configured to generate an IEGM signal based on electrical activity of the heart sensed by at least one electrode connected to the connector 110. The controller 130 could then process the IEGM signal to determine a current heart rate of the subject's heart.

Arrhythmia detection is traditionally performed by an IMD 100 if the current heart rate exceeds a predefined rate threshold. The IMD 100 could then use two different such rate thresholds, one rate threshold during the default pacing mode and another, typically lower rate threshold during the ischemic pacing mode.

The controller 130 advantageously switches back from the ischemic pacing mode to the default pacing mode after the end of the detected ischemic event. This switch back to the default pacing mode preferably occurs following a defined time period from when the ischemic region detector 132 detects the end of the ischemic event, i.e. when the constituent concentration in coronary venous blood has returned back to the normal concentration level at all sensor element sites. The length of this defined time period is preferably programmed into the IMD 100 by the subject's physician.

The IMD 100 as illustrated in FIG. 2 preferably comprises a ventricular sensing circuit 150 implemented in the IMD 100 to sense electrical activity in a ventricle using an electrode connected to the connector 110.

The IMD 100 may optionally also comprise a corresponding atrial pulse generator and an atrial sensing circuit (not shown in FIG. 2) if the connector 110 is connectable to at least one atrial cardiac lead. The ventricular and atrial pulse generators 140 of the IMD 100 may then include dedicated, independent pulse generators, multiplexed pulse generators, or shared pulse generators. The pulse generators 140 are controlled by the controller 130 via appropriate control signals, respectively, to trigger or inhibit the stimulating pulses.

The ventricular and optional atrial sensing circuits 150 of the IMD 100 may include dedicated sense amplifiers, multiplexed amplifiers, or shared amplifiers. The electronic configuration switch 120 determines the “sensing polarity” of the cardiac signal by selectively closing the appropriate switches, as is also known in the art. In this way, the physician may program the sensing polarity independent of the stimulation polarity. The sensing circuits are optionally capable of obtaining information indicative of tissue capture.

Each sensing circuit 150 preferably employs one or more low power, precision amplifiers with programmable gain and/or automatic gain control, band-pass filtering, and a threshold detection circuit, as known in the art, to selectively sense the cardiac signal of interest.

The outputs of the ventricular and optional atrial sensing circuits 150 are connected to the controller 130, which, in turn, is able to trigger or inhibit the ventricular and optional atrial pulse generators 140, respectively, in a demand fashion in response to the absence or presence of cardiac activity in the appropriate chambers of the heart.

The controller 130 of the IMD 100 is preferably in the form of a programmable microcontroller 130 that controls the operation of the IMD 100. The controller 130 typically includes a microprocessor, or equivalent control circuitry, designed specifically for controlling the delivery of pacing therapy, and may further include RAM or ROM memory, logic and timing circuitry, state machine circuitry, and I/O circuitry. Typically, the controller 130 is configured to process or monitor input signals as controlled by a program code stored in a designated memory block. The type of controller 130 is not critical to the described implementations. In clear contrast, any suitable controller may be used that carries out the functions described herein. The use of microprocessor-based control circuits for performing timing and data analysis functions are well known in the art.

Furthermore, the controller 130 is also typically capable of analyzing information output from the sensing circuits 150 to determine or detect whether and to what degree tissue capture has occurred and to program a pulse, or pulse sequence, in response to such determinations. The sensing circuits 150, in turn, receive control signals over signal lines from the controller 130 for purposes of controlling the gain, threshold, polarization charge removal circuitry, and the timing of any blocking circuitry coupled to the inputs of the sensing circuits 150 as is known in the art.

The optional electronic configuration switch 120 includes a plurality of switches (not shown) for connecting the desired connector terminals 111-117 to the appropriate I/O circuits, thereby providing complete electrode programmability. Accordingly, the electronic configuration switch 120, in response to a control signal from the controller 130, determines the polarity of the stimulating pulses by selectively closing the appropriate combination of switches as is known in the art.

While a particular IMD 100 is shown in FIG. 2, it is to be appreciated and understood that this is done merely for illustrative purposes. Thus, the techniques and methods described below can be implemented in connection with other suitably configured IMDs. Accordingly, the person skilled in the art can readily duplicate, eliminate, or disable the appropriate circuitry in any desired combination.

The IMD 100 additionally includes a battery 180 that provides operating power to all of the circuits shown in FIG. 2.

In FIG. 2, the ischemic region detector 132, the optional variability calculator 134, the optional message generator 136 and the optional mode selector 138 have been illustrated as being run by the controller 130. These units 132-138 can then be implemented as a computer program product stored in the memory 170 and loaded and run on a general purpose or specially adapted computer, processor or microprocessor, represented by the controller 130 in FIG. 2. The software includes computer program code elements or software code portions effectuating the operation of the units 132-138. The program may be stored in whole or part, on or in one or more suitable computer readable media or data storage means that can be provided in an IMD 100.

In an alternative approach, the units 132-138 are implemented as hardware circuits in the IMD 100, preferably connected to the controller 130, such as in the form of special purpose circuits, such as ASICs (Application Specific Integrated Circuits).

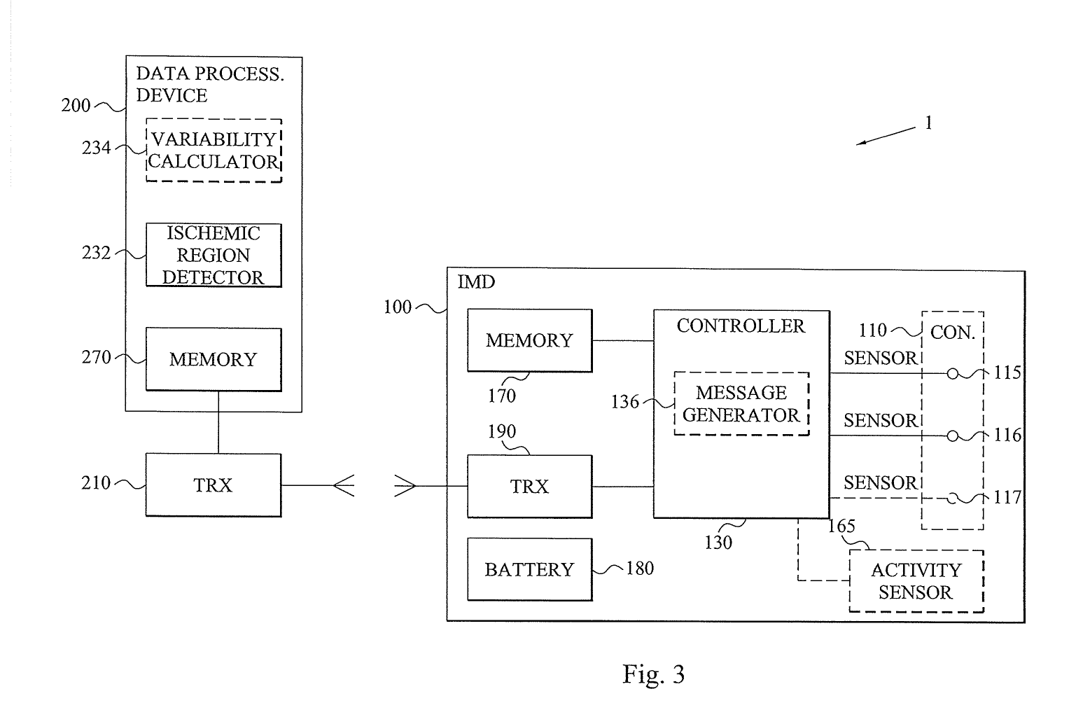

In the embodiment discussed above and disclosed in FIG. 2, the ischemia detecting system 1 is fully implantable and is housed in the IMD 100. In an alternative approach as shown in FIG. 3, the ischemia detecting system 1 is partly provided in the IMD 100 and partly provided in the non-implantable data processing device 200.

In such a case, the IMD 100 preferably provides the sensor signals as previously disclosed herein and stores them in the memory 170. Data representing these sensor signals, such as the sample values or a calculated average signal sample per sensor signal, is then composed by the message generator 136 into data packets that are transmitted by the transceiver 190 of the IMD 100 to a receiver or transceiver 210 of or connected to the data processing device 200. In this embodiment, the data processing device 200 comprises the ischemic region detector 232 and the optional variability calculator 234. The operation of these units 232, 234 are basically the same as previously disclosed herein.

In the embodiment of FIG. 3, the data processing device 200 preferably comprises or is connected to a display or screen (see FIG. 1) in order to visually present information of the detected ischemic region(s) to the subject or the subject's physician. The information generated by the ischemic region detector 232 can therefore be of high diagnostic value for the physician in order to assess the status and condition of the subject's heart. In particular, the generated information can be used to determine the ischemic burden of the heart, i.e. the frequency, duration and location of cardiac ischemic events. This information can therefore be used by the physician to determine appropriate treatment for the subject and can be used as an early warning for more severe heart conditions, such as myocardial infarctions and heart failure.

In particular, the information of ischemic heart regions can be used for titration of medicaments to the subject by monitoring the effect of medicament administration on cardiac ischemia. For instance, administration of correct amounts of medicaments to treat hypertension is not trivial. Too low levels of the medicament will not have the desired effect of reducing the blood pressure. However, if too high levels of the medicament is administered the blood pressure can be reduced too much so that blood supply in the coronary vascular system is not efficiently maintained and ischemia may occur. The information generated by the ischemic region detector 232 can therefore be used, among others, to verify correct levels of administered medicaments, such as anti-hypertensive drugs and anti-coagulants.

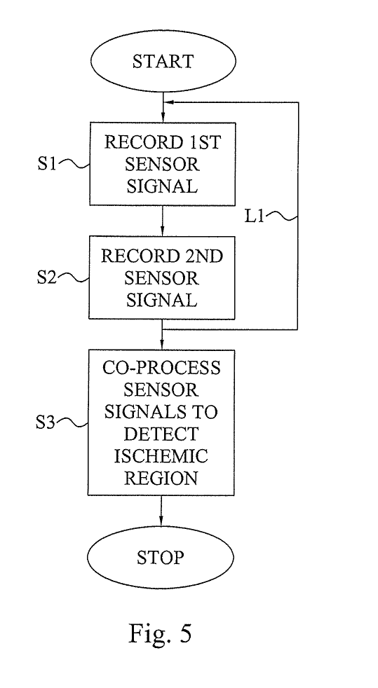

FIG. 5 is a flow diagram illustrating a method for detecting ischemia according to an embodiment. The method starts in step S1 were a first sensor signal representative of a concentration of a constituent in coronary venous blood in a first portion of the coronary venous system of a subject's heart is recorded at a site on a first side of a branching vein of the coronary venous system. Step S2 correspondingly records a second sensor signal representative of the concentration of the constituent in coronary venous blood in a second, different portion of the coronary venous system at a site on a second, opposite side of the branching vein. Steps S1 and S2 can be performed serially in any order or at least partly in parallel.

The recording of the sensor signals are preferably performed during a predefined time or until a predefined number of signal samples have been obtained as indicated by the line L1.

The sensor signals recorded in steps S1 and S2 are then co-processed in step S2 to detect an ischemic heart region based on a relation between the sensor signals as previously disclosed herein.

Although modifications and changes may be suggested by those skilled in the art, it is the intention of the inventors to embody within the patent warranted hereon all changes and modifications as reasonably and properly come within the scope of their contribution to the art.

We claim as our invention:

1. An ischemia detecting system comprising:
an implantable medical device comprising a connector connectable to a left ventricular cardiac catheter comprising at least a first sensor element and a second sensor element arranged on said left ventricular cardiac catheter to be positioned on either side of a branching vein in a coronary venous system of a heart in a subject, said first sensor element constituting a first sensor, configured to generate a first sensor signal representative of a concentration of a constituent in coronary venous blood in a first portion of said coronary venous system, or is connectable to a common sensor configured to generate, for said first sensor element, said first sensor signal, and said second sensor element constituting a second sensor, configured to generate a second sensor signal representative of a concentration of said constituent in coronary venous blood in a second portion of said coronary venous system, or is connectable to said common sensor configured to generate, for said second sensor element, said second sensor signal, and a memory configured to store said first sensor signal and said second sensor signal; and
an ischemic region detector configured to co-process said first sensor signal and said second sensor signal to detect an ischemic region of said heart based on a relation between said first sensor signal and said second sensor signal.

2. The ischemia detecting system according to claim 1, wherein said ischemic region detector is configured to detect said ischemic region based on a relation between said concentration of said constituent in coronary venous blood in said first portion of said heart represented by said first sensor signal and said concentration of said constituent in coronary venous blood in said second portion of said heart represented by said second sensor signal.

3. The ischemia detecting system according to claim 1, wherein said ischemic region detector is configured to detect said ischemic region by comparing a quotient or a difference between said concentration of said constituent in coronary venous blood in said first portion of said heart represented by said first sensor signal and said concentration of said constituent in coronary venous blood in said second portion of said heart represented by said second sensor signal with a threshold value.

4. The ischemia detecting system according to claim 1, further comprising a variability calculator configured to i) calculate, based on said first sensor signal, a first variability value representing a variability in said concentration of said constituent in coronary venous blood in said first portion of said heart represented by said first sensor signal and ii) calculate, based on said second sensor signal, a second variability value representing a variability in said concentration of said constituent in coronary venous blood in said second portion of said heart represented by said second sensor signal, wherein said ischemic region detector is configured to detect said ischemic region based on a relation between said first variability value and said second variability value.

5. The ischemia detecting system according to claim 1, wherein said connector is connectable to said left ventricular cardiac catheter configured to be implanted into a posterior vein of a left ventricle of said heart and said first sensor element is configured to be positioned in a coronary sinus of said heart and said second sensor element is configured to be positioned in said posterior vein.

6. The ischemia detecting system according to claim 1, wherein said connector is connectable to said left ventricular cardiac catheter configured to be implanted into a lateral vein of a left ventricle of said heart and said first sensor element is configured to be positioned in one of a coronary sinus and a great cardiac vein of said heart and said second sensor element is configured to be positioned in said lateral vein.

7. The ischemia detecting system according to claim 6, wherein:
said connector is connectable to said left ventricular cardiac catheter further comprising a third sensor element arranged on said left ventricular cardiac catheter, said third sensor element constituting a third sensor, configured to generate a third sensor signal representative of a concentration of said constituent in coronary venous blood in a third portion of said coronary venous system, or is connectable to said common sensor configured to generate, for said third sensor element, said third sensor signal, and said first sensor element is configured to be positioned in said coronary sinus upstream of a posterior vein of said left ventricle and said third sensor element is configured to be positioned in one of said coronary sinus and said great cardiac vein downstream of said posterior vein;
said memory is configured to store said first sensor signal, said second sensor signal and said third sensor signal; and
said ischemic region detector is configured to co-process said first sensor signal, said second sensor signal and said third sensor signal to detect said ischemic region of said heart based on a relation between said first sensor signal, said second sensor signal and said third sensor signal.

8. The ischemia detecting system according to claim 1, wherein said connector is connectable to said left ventricular cardiac catheter configured to be implanted in an anterior interventricular vein of said heart and said first sensor element is configured to be positioned in one of a coronary sinus and a great cardiac vein of said heart and said second sensor element is configured to be positioned in said anterior interventricular vein.

9. The ischemia detecting system according to claim 8, wherein:
said connector is connectable to said left ventricular cardiac catheter further comprising a third sensor element arranged on said left ventricular cardiac catheter, said third sensor element constituting a third sensor, configured to generate a third sensor signal representative of a concentration of said constituent in coronary venous blood in a third portion of said coronary venous system, or is connectable to said common sensor configured to generate, for said third sensor element, said third sensor signal, and said first sensor element is configured to be positioned in said coronary sinus upstream of a posterior vein of said left ventricle and said third sensor element is configured to be positioned in one of said coronary sinus and said great cardiac vein downstream of said posterior vein;
said memory is configured to store said first sensor signal, said second sensor signal and said third sensor signal; and
said ischemic region detector is configured to co-process said first sensor signal, said second sensor signal and said third sensor signal to detect said ischemic region of said heart based on a relation between said first sensor signal, said second sensor signal and said third sensor signal.

10. The ischemia detecting system according to claim 8, wherein:
said connector is connectable to said left ventricular cardiac catheter further comprising a third sensor element arranged on said left ventricular cardiac catheter, said third sensor element constituting a third sensor, configured to generate a third sensor signal representative of a concentration of said constituent in coronary venous blood in a third portion of said coronary venous system, or is connectable to said common sensor configured to generate, for said third sensor element, said third sensor signal, and said first sensor element is configured to be positioned in one of said coronary sinus and said great cardiac vein upstream of a lateral vein of said left ventricle and said third sensor element is configured to be positioned in said great cardiac vein downstream of said lateral vein;
said memory is configured to store said first sensor signal, said second sensor signal and said third sensor signal; and
said ischemic region detector is configured to co-process said first sensor signal, said second sensor signal and said third sensor signal to detect said ischemic region of said heart based on a relation between said first sensor signal, said second sensor signal and said third sensor signal.

11. The ischemia detecting system according to claim 1, wherein:
said connector is connectable to said left ventricular cardiac catheter comprising N sensors elements arranged on said left ventricular cardiac catheter to be positioned at different sites in said coronary venous system, each sensor element of said N sensor elements constitutes a respective sensor configured to generate a sensor signal representative of a concentration of said constituent in coronary venous blood in a respective portion of said coronary system, or is connectable to said common sensor configured to generate, for said sensor element, said sensor signal, N is an integer equal to or larger than three;
said memory is configured to store said N sensor signals; and
said ischemic region detector is configured to co-process said N sensor signals to detect said ischemic region of said heart based on a relation between said N sensor signals.

12. The ischemia detecting system according to claim 1, wherein said first sensor and said second or said common sensor is configured to generate a sensor signal representative of oxygen concentration in coronary venous blood and is selected from an implantable partial oxygen pressure, pO₂, sensor or an implantable oxygen saturation, SO₂, sensor.

13. The ischemia detecting system according to claim 1, wherein said first sensor element is a first sensor configured to generate said first sensor signal and said second sensor element is a second sensor configured to generate said second sensor signal.

14. The ischemia detecting system according to claim 1, wherein said first sensor element and said second sensor element are connected to said common sensor arranged on said left ventricular cardiac catheter, said common sensor is connectable to said connector.

15. The ischemia detecting system according to claim 1, wherein said first sensor element and said second sensor element are connectable to said common sensor arranged in said implantable medical device.

16. The ischemia detecting system according to claim 1, further comprising an activity sensor configured to generate an activity signal representative of a current activity level of said subject, wherein said ischemic region detector is configured to co-process said first sensor signal and said second sensor signal to detect said ischemic region of said heart if said current activity level of said subject as defined by said activity signal exceeds an activity threshold level.

17. The ischemia detecting system according to claim 1, wherein said implantable medical device comprises said ischemic region detector.

18. The ischemia detecting system according to claim 17, wherein said left ventricular cardiac catheter is a left ventricular cardiac lead comprising at least one pacing electrode and said implantable medical device further comprises:
a pulse generator connected to said connector and configured to generate pacing pulses to be applied to at least one pacing electrode of said left ventricular cardiac lead; and
a pulse generator controller connected to said pulse generator and configured to control said pulse generator to apply pacing pulses to said at least one pacing electrode according to a default pacing mode or an ischemic pacing mode, wherein said pulse generator controller is configured to switch from said default pacing mode to said ischemic pacing mode based on said ischemia region detector detecting said ischemic region.

19. The ischemia detecting system according to claim 1, wherein said implantable medical device further comprises a transmitter configured to transmit information of said first sensor signal and said second signal to an non-implantable data processing device comprising a receiver configured to receive said information, said non-implantable data processing device comprises said ischemic region detector.

20. A method for detecting ischemia comprising:
recording a first sensor signal representative of a concentration of a constituent in coronary venous blood in a first portion of a coronary venous system of a heart in a subject at a site on a first side of a branching vein of said coronary venous system;
recording a second sensor signal representative of a concentration of said constituent in coronary venous blood in a second portion of said coronary venous system at a site on a second, opposite side of said branching vein of said coronary venous system;
in a computerized processor, co-processing said first sensor signal and said second sensor signal to detect an ischemic region of said heart based on a relation between said first sensor signal and said second sensor signal, and providing an output from said processor in electronic form that includes said ischemic region.
